(19) United States
(12) Patent Application Publication
(10) Pub. No.: US 2016/0129065 A1
(43) Pub. Date: May 12, 2016

(54) COMPOSITION FOR PREVENTING AND TREATING GASTROINTESTINAL DISEASES, CONTAINING ESSENTIAL OILS EXTRACTED FROM LITSEA JAPONICA FRUIT

(71) Applicants: HURUM CO.,LTD., Seoul (KR); JEJU TECHNOPARK, Jeju-do (KR)

(72) Inventors: Ju-Hyun CHO, Chungju (KR); In-Jae PARK, Cheonan (KR); Yong-Hwan JUNG, Seogwipo (KR); Goo-Hee CHOI, Cheongju (KR); Weon-Jong YOON, Jeju (KR); Sung-Hwan PARK, Jeju (KR); Ji-Hyun YUN, Jeju (KR); Soon-Ok BAIK, Incheon (KR)

(21) Appl. No.: 14/899,323

(22) PCT Filed: Apr. 30, 2014
(86) PCT No.: PCT/KR2014/003894

§ 371 Date: Dec. 17, 2015

(30) Foreign Application Priority Data
Jun. 24, 2013 (KR) 10-2013-0072363

(51) Int. Cl. A61K 36/54

(52) U.S. Cl. CPC A61K 36/54

(57) ABSTRACT

The present invention relates to a composition for preventing and treating gastrointestinal diseases by using Litsea japonica, and specifically, to a composition for preventing and treating gastrointestinal diseases, containing essential oils extracted from Litsea japonica fruit as an active ingredient. According to the present invention, the essential oils extracted from Litsea japonica fruits (pure fruit, seed fruit, and seeds) and used as the active ingredient of the composition for preventing and treating gastrointestinal diseases have been confirmed to have excellent effects in an efficacy evaluation for relieving gastrointestinal diseases caused by Helicobacter pylori, which is a gastrointestinal disease factor, physical factors induced by stress, and chemical factors induced by indomethacin. Therefore, the essential oils extracted from Litsea japonica fruit (pure fruit, seed fruit, and seeds) are useful for preventing and treating gastrointestinal diseases caused by various factors.

### TECHNICAL FIELD

The present invention relates to a composition for preventing and treating gastrointestinal diseases using Litsea japonica, and in particular to a composition for preventing and treating gastrointestinal diseases, containing essential oils extracted from Litsea japonica fruit as an active ingredient.

### BACKGROUND ART

In case of Korea, the medical care utilization rate of outpatients suffering from gastrointestinal diseases in 1999 was 1156 persons per 100000 persons, which is the highest medical care utilization rate followed by respiratory diseases of 1156 persons (Ministry of Health and Welfare 2001), and in case of gastrointestinal diseases, for example, stomach and duodenum ulcers, colorectal cancer, etc. in 2001 in Korea, the death rate due to gastrointestinal diseases was 30.1 persons per 100000 persons, which is a high rate (Statistics Korea, 2002).

The gastrointestinal diseases may be caused by various factors, for example, bacteria-associated diseases, a stress, medicines such as ethanol, etc., changes in food and life patterns, aging, etc. For this reason, the gastrointestinal diseases continue to increase.

The causes of the gastrointestinal diseases may be categorized into a bacteria-associated disease factor, for example, Helicobacter pylori, etc., a physical factor, for example, a stress, etc., and a chemical factor, for example, ethanol, etc.

The major symptoms of gastrointestinal diseases (gastritis, gastric ulcer, duodenal ulcer, etc.) are abdominal pain, heartburn, bloated, dyspepsia, burp, nausea, vomiting, bleeding, etc. In order to alleviate such symptoms, people in general take antacid for quick effects. This however shows temporal effects. The uses of steroid and nonsteroidal antiinflammatory analgesic drugs are restricted due to their side effects. In particular, the uses of nonsteroidal antiinflammatory analgesic drugs, for example, aspirin, ibuprofen, etc. are known to aggravate gastric ulcer or duodenal ulcer, so such medicines are extremely inhibited from uses for the patients who are suffering from ulcer, or it is recommended that such drugs are taken together with antacid. To this end, it needs to develop a new medicine which has fewer side effects and is able to protect stomach even when it is administrated for a long time, while alleviating any symptom which may occur in gastrointestinal diseases.

The Litsea japonica used in the present invention is an evergreen leaf arbor and in general grows in a seaside area or at the foot of a mountain lower than 700 m altitude in a vertical direction.

In the previous studies on the chemical ingredients of this plant, it was reported that various kinds of essential oils, fatty acids, lactones, alkaloids and terpenoids were found.

Hamabiwalactone A, hamabiwalatone B, akolactone B, litsealactone A and litsealactone B which were known as the kinds of Lactones, were separated.

As a result of the studies conducted on the anti-complement activity of epicatechin, afzelin, quercitrin and tiliroside which are the ingredients of the flavonoids separated from the leaves, it was reported that the tilirosie had the strongest inhibition effects on the complement system.

As a result of the studies wherein 80% EtOH extract was fractionated from the leaves of Litsea japonica, and the antioxidant activity (DPPH and superoxide radical inhibition activity was good at DPPH, Xanthine Oxidase, Superoxide radical->fraction EtOAC layer) and LDH, NO, PGE₂ (saline) were measured using the fractions (Yoon, Won-jong et. al., 2010, J, Korean Soc. Appl. Biol. Chem. 53(1): p 27˜32), and the activity of the anti-complement system was measured after the flavonoid of leaves was separated (Epicatechin, Afzlin, Quercitrin, Tiliroside, Myricitrin, Kaempferol, Quercetin, Myricetin) using a silica column (Sun Young Lee, et. al., 2005, PHYTOTHERAPY RESEARCH, 19: p. 273-276), and the Litsea japonica among edible and medical plants was extracted using methanol, and lipid peroxide generation inhibition was measured (Park, Jonh-chul et. al., 1997, J. Korean Soc. Food Sci. Nutr. 26(6): p. 1159-1163), it was reported that the activity in case of the use of Litsea japonica showed about 18% inhibition effects at a concentration of 10⁻¹ mg/m L.

The studies on Litsea japonica are partially conducted, for example, it was reported that a study was conducted on the induction effects of the HL-60 leukemia cell apoptosis of Litsea japonica (2009, Kim Elvira, et. al., Ykhak Hoeji, 53(1): p. 6-11), wherein 80% ethanol was extracted using the leaves of Litsea japonica, and the normal cells showed no any toxicity as a result of the measurement of the HL-60 (cell toxicity)-MTT, and it showed a HL-60 (leukemia cell) cell proliferation inhibition effect, and through the DNA fragmentation analysis and the Western blot analysis, it was confirmed that a Bcl-2 expression was decreased, and a proliferation inhibition effect was obtained by inducing the apoptosis by activating the caspase.

As the patent document prior art technology, a technology on a Litsea japonica extract which shows an anti-inflammation activity and a bone metabolism-associated factor inhibition activity is disclosed in the Korean patent publication number 10-2008-0020738 (the publication date: Mar. 6, 2008), and a technology on a composition for preventing and treating a cancer disease containing the extract of Litsea japonica as an active ingredient is disclosed in the Korean patent publication number 10-2009-0091477 (the publication date: Aug. 28, 2009), and a technology on a composition for preventing and treating a diabetes mellitus complication containing the extract of Litsea japonica and its fractions as an active ingredient is disclosed in the Korean patent registration number 10-1243243 (the registration date: Mar. 7, 2012).

### SUMMARY OF THE INVENTION

### Disclosure of the Invention

Accordingly, it is an object of the present invention to provide a composition for preventing and treating gastrointestinal diseases which may satisfy the needs for a new medicine development while alleviating symptoms which occur in gastrointestinal diseases different from a conventional medicine (substance) used for preventing and treating gastrointestinal diseases and protecting stomach with less side effects even though it is used for a long time.

It is another object of the present invention to provide a composition for preventing and treating gastrointestinal diseases, containing essential oils extracted from Litsea japonica fruit as an active ingredient, wherein the applicant of the present application has confirmed that the essential oils extracted from Litsea japonica fruit (pure fruit, seed fruit or seeds) have effects on the diseases which may lead to various gastrointestinal diseases (bacteria-associated cause, physical cause or chemical cause) while the applicant studies natural substances in order to develop a material which has effect on a prevention and treatment of gastro-intestinal track disease.

### Technical Solution

To achieve the above objects, there is provided a composition for preventing and treating gastrointestinal diseases which may include essential oils extracted from the fruit of Litsea japonica.

The essential oils are extracted from any of the pure fruit (seeds are removed), seed fruit (seeds are included) and seeds (seeds of fruit) of Litsea japonica.

The essential oils are formed of a pure fruit essential oil extracted from the pure fruit of Litsea japonica, and a seeds essential oil extracted from the seeds of Litsea japonica, and the pure fruit essential oils and the seeds essential oils can be used in singular forms or can be used in a combination thereof.

The essential oils are extracted using any of an alcohol extraction method, a hexane extraction method, a super critical extraction method, a distillation method, a compression method, and an ultra high pressure extraction method.

As for the method for preparing essential oils extracted from the fruit of Litsea japonica for a composition which is able to prepare and treat gastrointestinal diseases, the essential oils may be prepared by a method including a process wherein an extraction is carried using 95% alcohol (EtOH) with respect to the pure fruit or seeds of Litsea japonica, and the alcohol is removed, and a layer is separated by inputting water or alternatively, the essential oils may be obtained by using a synthetic absorbent, or the essential oils may be obtained in such a way that a normal hexane (n-hexane), which is a solvent, is inputted 9˜11 times as compared to the weight thereof into the pure fruit or seeds of Litsea japonica, and the mixture is stirred for 24 hours at a room temperature, and the extraction is carried, and a hexane layer is removed.

### Advantageous Effects

The essential oils extracted from the Litsea japonica fruit according to the present invention contain ingredients, for example, Beta-cubebene, Beta-selinene, Gamma-Elemene, Beta-Caryophyllene, etc., and as a result of the experiments, it was confirmed that the essential oils of the present invention have a good anti-Helicobacter effect, a function improvement effect on gastrointestinal diseases, a function improvement effect on gastrointestinal diseases which may occur due to indomethacin (chemical cause), etc., so the essential oils extracted from Litsea japonica fruit according to the present invention can be used as an active ingredient of a composition for preventing and treating stomach-related diseases.

### BRIEF DESCRIPTION OF DRAWINGS

FIG. 1 is a graph showing an improvement effect of essential oils extracted from Litsea japonica fruit on gastrointestinal diseases caused due to stress according to an exemplary embodiment of the present invention.

FIG. 2 is a graph showing an improvement effect of essential oils extracted from Litsea japonica fruit on gastrointestinal diseases caused due to indomethacin according to an exemplary embodiment of the present invention.

FIG. 3 is a graph showing a gastrointestinal diseases inhibition rate of essential oils extracted from Litsea japonica fruit on a stress cause (water-immersion-restraint model) and a chemical cause (indomethacin) according to an exemplary embodiment of the present invention.

FIG. 4 is a chromatogram graph showing essential oils extracted from Litsea japonica fruit according to a first exemplary embodiment of the present invention.

FIG. 5 is a chromatogram graph showing essential oils extracted from Litsea japonica fruit according to a second exemplary embodiment of the present invention.

### BEST MODES FOR CARRYING OUT THE INVENTION

The present invention provides a composition for preventing and treating gastrointestinal diseases and a method for preparing the same which can be used to prevent or treat gastrointestinal diseases, wherein the composition contains essential oils extracted from Litsea japonica fruit as an active ingredient.

The present invention will be described in detail below.

The essential oils extracted from Litsea japonica fruit which is an active ingredient of the composition according to the present invention can be extracted from fruit (hereinafter referred to “pure fruit”) from which seeds are removed, and seeds (seeds within fruit), and essential oils are obtained from the same. Such essential oils can be prepared by the method below.

It is possible to obtain essential oils from the fruit (hereinafter referred to “seed fruit”) including the seeds, from which the seeds are not removed, without separating the Litsea japonica fruit into the pure fruit and the seeds.

The present embodiment of the present invention is directed to obtaining essential oils by separating the pure fruit and seeds in an effort to seek any application of various compositions, for example, medicine and medical supplies, food, etc., and alternatively it is possible to obtain essential oils from the seed fruit (the fruit containing seeds), which is also included in the scope of rights of the present invention.

The method for extracting essential oils from Litsea japonica fruit (pure fruit, seeds or seed fruit) may be any of an alcohol (EtOH) extraction method, a super critical extraction method, a distillation method, a compression method, a hexane extraction method, an ultra high pressure extraction method, etc.

In case where the distillation method and the compression method are used, it is disadvantageous that the quantity of the essential oils is small. In case where the hexane extraction method is used, which is an extraction method using hexane, it is good in terms of the extraction of essential oils as compared with other extraction methods. The alcohol extraction method which allows to extract via step-by-step treatments from has also good advantages.

Step-by-Step Extraction Method 1 Using Alcohol (Essential Oils of Pure Fruit of Litsea japonica)

The extraction on the dried pure fruit of Litsea japonica is carried out using 95% of alcohol which is solvent, and the alcohol was removed, and essential oils were obtained using a HP-20 resin which is a synthetic adsorbent (when 100 g of the pure fruit of Litsea japonica was used, 15 g of essential oils was obtained).

Step-by-Step Extraction Method 2 Using Alcohol (Essential Oils of Pure Fruit of Litsea japonica)

The essential oils were obtained via a step-by-step process wherein the extraction on the dried pure fruit of Litsea japonica is carried out using 95% of alcohol which is solvent, and the alcohol was removed, and it was immersed in water, thus separating layers (when 100 g of the pure fruit of Litsea japonica was used, 17 g of essential oils was obtained).

Extraction Method Using Hexane (Essential Oils of Pure Fruit of Litsea japonica)

9˜11 times (preferably 10 times) of the normal hexane (n-hexane), which is a solvent, as compared with the weight thereof, was inputted into the dried pure fruit of the Litsea japonica, and the extraction was carried out by stirring it for 24 hours at a room temperature (15˜25° C.), and the hexane layer was removed, thus obtaining the essential oils of the Litsea japonica fruit.

Extraction Method Using Hexane (Essential Oils of Seeds of Litsea japonica)

9˜11 times (preferably 10 times) of the normal hexane (n-hexane), which is a solvent, as compared with the weight thereof was inputted into the dried seeds of the Litsea japonica, and the extraction was carried out by stirring it for 24 hours at a room temperature (15˜25° C.), and the hexane layer was removed, thus obtaining the essential oils of the Litsea japonica seeds.

The obtained essential oils of the pure fruit of Litsea japonica and the obtained essential oils of the seeds of the Litsea japonica can be used in a singular form or may be use after they are mixed at a predetermined ratio.

The essential oils extracted from the pure fruit and seeds of the Litsea japonica have good effects on the improvements of gastrointestinal diseases which may come from Helicobacter pylori which is a cause of gastrointestinal diseases, a physical cause (stress) and a chemical cause (indomethacin).

### MODES FOR CARRYING OUT THE INVENTION

### First Exemplary Embodiment

### Preparation of Essential Oils from Pure Fruit of Litsea japonica

The process was carried out step-by-step in such a way that 5000 mL of 95% alcohol was added to 500 g of the frozen and dried pure fruit of Litsea japonica, and the mixture was stirred and extracted for 24 hours at a room temperature (20° C.), and then the alcohol was removed, and water was added, thus obtaining 85 g of essential oils of the pure fruit of the Litsea japonica.

### Second Exemplary Embodiment

### Preparation of essential oils from pure fruit of Litsea japonica

5000 mL of normal hexane (n-hexane) was added to 500 g of the frozen and dried pure fruit of Litsea japonica, and the mixture was stirred and extracted for 24 hours at a room temperature (20° C.), and then the hexane was removed, thus obtaining 95 g of essential oils of the pure fruit of the Litsea japonica.

### Third Exemplary Embodiment

### Preparation of Essential Oils from Sees of Litsea japonica

5000 mL of normal hexane (n-hexane) was added to 500 g of the frozen and dried seeds of Litsea japonica, and the mixture was stirred and extracted for 24 hours at a room temperature (20° C.), and then the hexane was removed, thus obtaining 46 g of essential oils of the seeds of the Litsea japonica.

### Fourth Exemplary Embodiment

### Preparation of Essential Oils from Pure Fruit of Litsea japonica

5000 mL of 95% alcohol (EtOH) was added to 500 g of the frozen and dried pure fruit of Litsea japonica, and the mixture was stirred and extracted for 24 hours at a room temperature (20° C.), and then the alcohol was removed, and a HP-20 resin, which is a synthetic adsorbent, was used step-by-step, thus obtaining 75 g of essential oils of the pure fruit of the Litsea japonica.

### Experiment Example 1

### Ingredient Analysis on Essential Oils from Pure Fruit of Litsea japonica

The analysis experiment was carried out on the essential oils from the pure fruit of Litsea japonica obtained according to the first and second exemplary embodiments.

Experiment method: The tool was Shimadzu, QP2010Plus, and the column was RTX-5 (30 m×0.25 mm, 0.25 μm), and for the other conditions, the analysis was carried under the conditions where the injection temperature was 250° C., and the detector temperature was 250° C., and the mass range (m/z) was 30-400, and the injection volume was 1 μL.

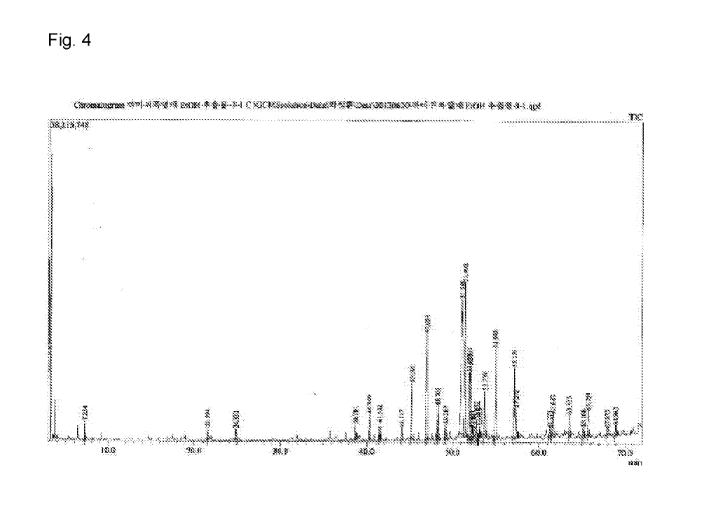

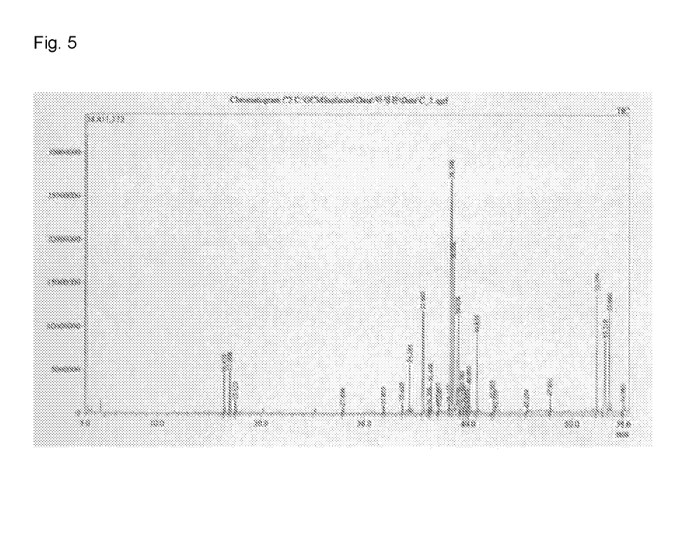

Experiment result: A result shown in FIGS. 4 and 5 and Tables 1 and 2 was obtained by analyzing the essential oils from pure fruit of Litsea japonica obtained according to the first and second exemplary embodiments.

The essential oils from the pure fruit of the first exemplary embodiment contained Beta-cubebene (19.7±2.4%), Beta-selinene (12.1±3.1%), and Beta-Caryophyllene (8.2±1.2%).

The essential oils from the pure fruit of the second exemplary embodiment contained Beta-cubebene (21.7±1.9%), Beta-selinene (11.1±1.8%), and Beta-Caryophyllene (7.4±1.6%).

TABLE 1
Analysis table on essential oils from pure fruit of Litsea japonica
obtained according to the first exemplary embodiment

| Compound Name | Area(%) | peak | Remarks |
|---|---|---|---|
| beta-cubebene | 19.7 ± 2.4 | 12 | |
| beta-selinene | 12.1 ± 3.1 | 13 | |
| beta-Caryophyllene | 8.2 ± 1.2 | 9 | |

TABLE 2
Analysis table on essential oils from pure fruit of Litsea japonica
obtained according to the second exemplary embodiment

| Compound Name | Area(%) | peak | Remarks |
|---|---|---|---|
| beta-cubebene | 21.7 ± 1.9 | 14 | |
| beta-selinene | 11.1 ± 1.8 | 15 | |
| beta-Caryophyllene | 7.4 ± 1.6 | 8 | |

### Experiment Example 2

### Stomach Function Improvement Effects of Essential Oils from Fruit of Litsea japonica (1): Anti-Helicobacter Effects

The experiment was carried out by a paper disk method so as to confirm the gastro-intestinal track diseases improvement effects of the essential oils from the pure fruit and the seeds of the Litsea japonica obtained according to the above exemplary embodiments.

Experiment method: The antimicrobial activity of each extract with respect to H. pylori was experimented by the paper disc method (an agar diffusion method). A test solution was absorbed by 50 μL in a bacteria free method into a 10 mm paper disc (ADVANTEC, Japan), which had been dried in a dry oven at 90° C., and was dried. The H. pylori which had been cultured in the agar medium was subjected to suspension in sterilized water to be over 1×10⁸ CFU (Colony Forming Unit), thus obtaining a strain liquid. The strain liquid was spread by 100 μL in an agar medium to which antibiotics was not added, and the paper disc dried by absorbing the test solution was attached to the top of the agar medium on which the H. pylori was spread. It was cultivated for 72 hours in a non-aerobic state at 37° C., and the size (size, mm) of the growth inhibition zone (clear zone) formed near the paper disc while the H. pylori was growing, was measured.

Experiment result: The sizes of the growth inhibition zones with respect to two kinds of Helicobacter pylori bacteria (KCTC12083 and KCTC 5335) on the essential oils obtained from the pure fruit and the seeds of Litsea japonica according to the above exemplary embodiments were measured and shown in Table 3. As seen in the following table 3, the essential oils obtained from the pure fruit and the seeds of Litsea japonica have good effects on the anti-Helicobacter improvements.

TABLE 3
The sizes of the growth inhibition zones of the Helicobacter pylori
bacteria (KCTC12083, KCTC5335) of the essential oils from the
pure fruit and seeds of Litsea japonica.

Sizes of growth inhibition
zones (mm)

| Samples | KCTC12083 | KCTC5335 |
|---|---|---|
| Control group(distilled water) | 0 | 0 |
| First embodiment (essential oils | 26 | 25 |
| from pure fruit) | | |
| Second embodiment (essential oils | 23 | 20 |
| from pure fruit) | | |
| Third embodiment (essential oils | 20 | 19 |
| from seeds) | | |
| Fourth embodiment (essential oils | 28 | 26 |
| from pure fruit) | | |

### Experiment Example 3

Improvement Effects on Stomach Functions of Essential Oils from Fruit of Litsea japonica (Physical Cause).

In order to confirm the improvement effects on the gastro-intestinal track disease of the essential oils from the pure fruit of Litsea japonica obtained according to the first exemplary embodiment, an experiment was carried out so as to evaluate improvement effects on gastrointestinal diseases which was caused by stress which is one of physical causes among the causes of the gastrointestinal diseases.

Experiment method: A male SD rat (200˜220 g) was used. To the rats acclimated after purchase, the experiment substance, the medium control group and the positive control group (Stillen tab. 150 mg/kg) were orally administrated everyday once for seven days at a three-stage concentration consisting of a low concentration (20 mg/kg), a middle concentration (60 mg/kg) and a high concentration (120 mg/kg). The rat was constrained from eating food for 24 hours and was subjected to a restraint device, and the rat was immersed in water until an xiphoid process in the water tank of 22° C. and was induced to get the gastric ulcer. After the induction to the gastric ulcer, the rat was sacrificed, and the stomach was taken out, and 1% formalin (10 mL) was injected into the inside of the stomach, and the stomach was inputted in 1% formalin liquid and was fixed in place for one hour, and the stomach was incised in a vertical direction along a large curve portion, and the stuff in the stomach was removed and spread on a cork board, and the length (mm) of the gastric ulcer (bleeding portions) was measured using a stereoscopic microscope and was converted into score forms. The inhibition ratio of the gastrointestinal diseases was calculated using the formula 1.

Inhibition ratio (%)=(inflammation length of control group−inflammation length of experiment substance process group)/inflammation length of control group×100 [Formula 1]

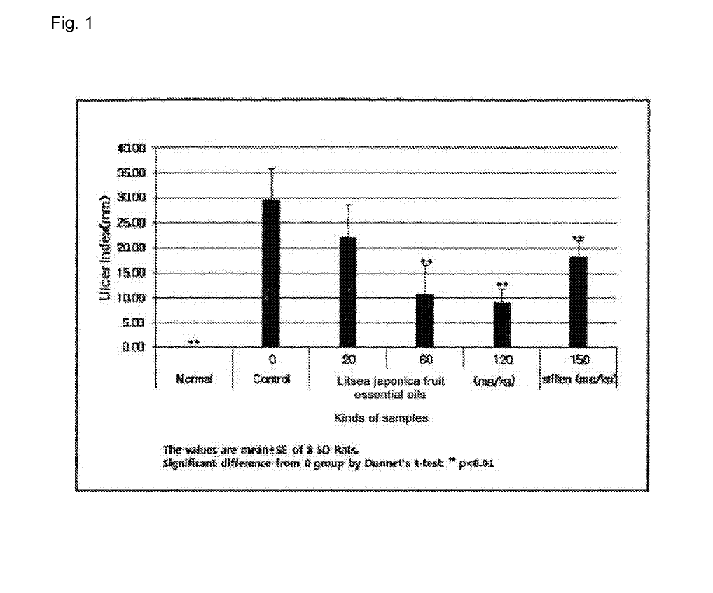

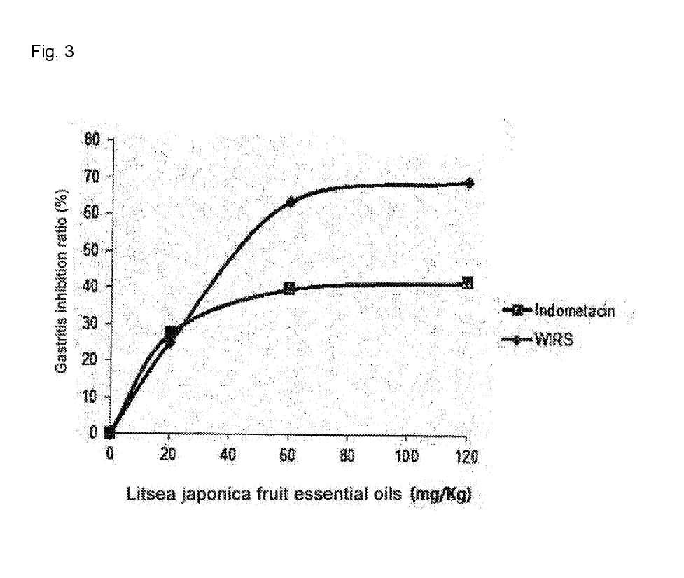

Experiment result: The ulcer index (mm) was shown in FIG. 1 and Table 4 by evaluating the improvement effects after the use of the essential oils from the fruit of Litsea japonica with respect to the gastrointestinal diseases induced by stress, and after the use of the essential oils from the fruit of Litsea japonica, the inhibition ratio of gastrointestinal diseases induced by the cause of the stress was shown in FIG. 3 and Table 5. Consequently, it was confirmed that the essential oils from the fruit of the Litsea japonica had improvement effects on the gastrointestinal diseases induced by stress.

The sample of the essential oils from the fruit of Litsea japonica showed 25.1% gastrointestinal diseases inhibition ratio at 20 mg/kg concentration.

The sample of the essential oils from the fruit of Litsea japonica showed 63.5% gastrointestinal diseases inhibition ratio at 60 mg/kg concentration.

69.2% gastrointestinal diseases inhibition ratio was obtained at the concentration of 120 mg/kg.

As compared with the fact that the Stillen tab. (150 mg/kg) which was the positive control group, showed 38.2% gastrointestinal diseases inhibition ratio, the essential oils from the fruit of Litsea japonica showed good effects on the improvements to the gastrointestinal diseases.

TABLE 4
The improvement effects on the gastrointestinal diseases via
gastritis index induced by a physical cause (stress) of the essential
oils from the pure fruit of Litsea japonica obtained according
to the first exemplary embodiment.

| | | Ulcer Index |
|---|---|---|
| Sample | | (mm) |
| Negative Control (Normal) | | 0 ± 0 |
| Positive Control (Stillen tab. 150 mg/kg) | | 18.3 ± 3.2 |
| Solvent control group | | 29.6 ± 6.2 |
| Essential oils from fruit | Low concentration (20 mg/kg) | 22.2 ± 6.4 |
| of Litsea japonica | Middle concentration (60 mg/kg) | 10.8 ± 5.9 |
| | High concentration (120 mg/kg) | 9.1 ± 3.2 |

TABLE 5
The gastrointestinal diseases inhibition ratio induced by a physical
cause (stress) of the essential oils from the fruit of Litsea japonica
obtained according to the first exemplary embodiment.

| | | Ulcer Index |
|---|---|---|
| Sample | | (mm) |
| Negative Control (Normal) | | — |
| Positive Control (Stillen tab. 150 mg/kg) | | 38.2 |
| Solvent control group | | 0 |
| Essential oils from fruit | Low concentration (20 mg/kg) | 25.1 |
| of Litsea japonica | Middle concentration (60 mg/kg) | 63.5 |
| | High concentration (120 mg/kg) | 69.3 |

### Experiment Example 4

### Improvement Effects on Stomach Functions of Essential Oils from Fruit of Litsea japonica: Improvement Effects on Gastrointestinal Diseases Induced by Indomethacin (Chemical Cause)

In order to confirm the improvement effects on the gastro-intestinal track disease of essential oils from the pure fruit of Litsea japonica obtained according to the first exemplary embodiment of the present invention, the experiments were carried out to evaluate the improvement effects on the gastrointestinal diseases induced by indomethacin which is a chemical cause among the causes inducing the gastrointestinal diseases.

Experiment method: A male SD rat (200˜220 g) was used. To the rats acclimated after purchase, the experiment substance, the medium control group and the positive control group (Stillen tab. 150 mg/kg) were orally administrated everyday once for seven days at a three-stage concentration consisting of a low concentration (20 mg/kg), a middle concentration (60 mg/kg) and a high concentration (120 mg/kg). The rat was constrained from eating food for 16 hours, and the indomethacin of 25 mg/kg (in 5% sodium bicarbonate) was orally administrated, and the gastric ulcer was induced, and the rat was sacrificed, and the stomach was taken out, and 1% formalin (10 mL) was injected into the inside of the stomach, and the stomach was inputted in 1% formalin liquid and was fixed in place for one hour, and the stomach was incised in a vertical direction along a large curve portion, and the stuff in the stomach was removed and spread on a cork board, and the length (mm) of the gastric ulcer (bleeding portions) was measured using a stereoscopic microscope and was converted into score forms. The inhibition ratio of the gastrointestinal diseases was calculated using the formula 2.

Inhibition ratio (%)=(inflammation length of control group−inflammation length of experiment substance process group)/inflammation length of control group×100 [Formula 2]

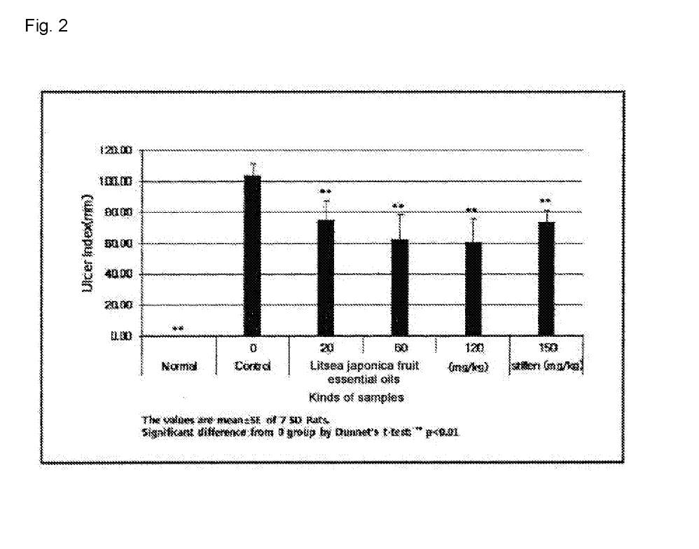

Experiment result: The ulcer index (mm) was shown in FIG. 2 and Table 6 by evaluating the improvement effects after the use of the essential oils from the fruit of Litsea japonica with respect to the gastrointestinal diseases induced by indomethacin, and after the use of the essential oils from the fruit of Litsea japonica, the inhibition ratio of gastrointestinal diseases induced by the cause of indomethacin was shown in FIG. 3 and Table 7. Consequently, it was confirmed that the essential oils from the fruit of the Litsea japonica had improvement effects on the gastrointestinal diseases induced by indomethacin which is a chemical cause.

The sample of the essential oils from the fruit of Litsea japonica showed 27.3% gastrointestinal diseases inhibition ratio at 20 mg/kg concentration.

The sample of the essential oils from the fruit of Litsea japonica showed 39.6% gastrointestinal diseases inhibition ratio at 60 mg/kg concentration.

41.8% gastrointestinal diseases inhibition ratio was obtained at the concentration of 120 mg/kg.

As compared with the fact that the Stillen tab. (150 mg/kg) which was the positive control group showed 30.0% gastrointestinal diseases inhibition ratio, the essential oils from the fruit of Litsea japonica showed good effects on the improvements to the gastrointestinal diseases which was induced by indomethacin.

TABLE 6
The gastrointestinal diseases improvement effects which were
shown with gastritis index induced by a chemical cause (indomethacin)
of the essential oils from the fruit of Litsea japonica obtained
according to the first exemplary embodiment.

| | | Ulcer Index |
|---|---|---|
| Sample | | (mm) |
| Negative Control (Normal) | | 0 ± 0 |
| Positive Control (Stillen tab. 150 mg/kg) | | 73.8 ± 7.4 |
| Solvent control group | | 103.9 ± 12.2 |
| Essential oils from fruit | Low concentration (20 mg/kg) | 75.5 ± 12.2 |
| of Litsea japonica | Middle concentration (60 mg/kg) | 62.8 ± 15.6 |
| | High concentration (120 mg/kg) | 60.5 ± 7.4 |

TABLE 7
The gastrointestinal diseases inhibition ratio induced by a chemical
cause (indomethacin) of the essential oils from the fruit of Litsea japonica
obtained according to the first exemplary embodiment.

| | | Ulcer Index |
|---|---|---|
| Sample | | (mm) |
| Negative Control (Normal) | | — |
| Positive Control (Stillen tab. 150 mg/kg) | | 30 |
| Solvent control group | | 0 |
| Essential oils from fruit | Low concentration (20 mg/kg) | 27.3 |
| of Litsea japonica | Middle concentration (60 mg/kg) | 39.6 |
| | High concentration (120 mg/kg) | 41.8 |

### INDUSTRIAL APPLICABILITY

The essential oils extracted from the Litsea japonica fruit (pure fruit, seed fruit or seeds) have good effects on the prevention and treatments of gastrointestinal diseases induced by a cause of Helicobacter pylori bacteria, a physical cause (stress) or a chemical cause (indomethacin).

To this end, the essential oils extracted from the fruit (pure fruit, seed fruit, seeds) of Litsea japonica according to the present invention can be usefully used for the prevention and treatment of gastrointestinal diseases which may be inducted by various causes.

1. A composition for preventing and treating gastrointestinal diseases, comprising: essential oils extracted from ea fruit of Litsea japonica.

2. The composition of claim 1, wherein the essential oils are extracted from any of the pure fruit, seed fruit and seeds of Litsea japonica.

3. The composition of claim 1, wherein the essential oils are formed of a pure fruit essential oil extracted from the pure fruit of Litsea japonica, and a seeds essential oil extracted from the seeds of Litsea japonica, and the pure fruit essential oils and the seeds essential oils can be used in singular forms or can be used in a combination thereof.

4. The composition of claim 1, wherein the essential oils are extracted using any of an alcohol extraction method, a hexane extraction method, a super critical extraction method, a distillation method, a compression method, and an ultra-high pressure extraction method.

5. The composition of claim 2, wherein the essential oils are extracted using any of an alcohol extraction method, a hexane extraction method, a super critical extraction method, a distillation method, a compression method, and an ultra-high pressure extraction method.

6. The composition of claim 3, wherein the essential oils are extracted using any of an alcohol extraction method, a hexane extraction method, a super critical extraction method, a distillation method, a compression method, and an ultra-high pressure extraction method.
